(19) United States
(12) Patent Application Publication
(10) Pub. No.: US 2022/0289652 A1
(43) Pub. Date: Sep. 15, 2022

(54) USE OF AROMATIC ALCOHOL AS STRUCTURAL INDUCER AND METHOD FOR TREATING SILK FIBROIN

(71) Applicant: SIMATECH INCORPORATION, Suzhou (CN)

(72) Inventors: Xiaoqin WANG, Suzhou (CN); Xusheng XIE, Suzhou (CN); Zhaozhu ZHENG, Suzhou (CN)

(21) Appl. No.: 17/630,398

(22) PCT Filed: Dec. 3, 2019
(86) PCT No.: PCT/CN2019/122715

§ 371 Date: Jan. 26, 2022

(30) Foreign Application Priority Data
Nov. 29, 2019 (CN) 201911201913.9

(51) Int. Cl. C07C 31/135; D01F 4/02; D01F 1/10; C07K 14/435

(52) U.S. Cl. CPC C07C 31/1355; D01F 4/02; D01F 1/10; C07K 14/43586; D10B 2211/04

(57) ABSTRACT

The present invention provides use of an aromatic alcohol as a structural inducer for silk fibroin, and also provides a method for treating silk fibroin. The method includes the following steps: bringing silk fibroin into contact with an aromatic alcohol-containing vapor or liquid to induce the structural transformation of silk fibroin, to obtain a water-insoluble silk fibroin, wherein the mechanical performance of the resulting silk fibroin material and the bonding strength between the silk fibroin material and a substrate material are over 50% higher than those of a silk fibroin material obtained by a traditional treatment method (induction with methanol, ethanol, and steam).

### FIELD OF THE INVENTION

The present invention relates to the technical field of materials, and more particularly to use of an aromatic alcohol as a structural inducer for silk fibroin and a method for treating silk fibroin materials.

### DESCRIPTION OF THE RELATED ART

Silk consists of outer sericin (20-30%) and inner silk fibroin (70-80%), from which regenerated silk fibroin can be obtained through dissolution, desalination and the like. Regenerated silk fibroin can be used to prepare materials in the form of particles, fibers, films, gels, tubes, scaffolds, and others. These materials are widely used many research fields such as the textile industry, light industry chemistry, drug control release carriers, and tissue engineering scaffolds.

Water-insoluble silk fibroin materials are usually obtained by physical methods such as pH, temperature, ultrasound, or vortex, chemical methods such as induction with a chemical reagent such as methanol, ethanol, and polyethylene glycol, etc., and chemically cross-linking with Genipin or HRP to realize the intramolecular and intermolecular cross-linking of free silk fibroin molecules.

Currently, a commonly used structural inducer for silk fibroin, such as steam, methanol, and ethanol, can be used to change the secondary structure of silk fibroin molecules in a dry silk fibroin material, to realize the intermolecular and intramolecular cross-linking through a physical force, thus making the material insoluble in water, and have strong mechanical performance and chemical and biological stability.

These methods still need improvement. For example, on the one hand, the mechanical performance (rigidity and toughness) of the prepared silk fibroin material is weak, and fails to meet the requirements in industry. Particularly silk fibroin materials are difficult to be formed into a stable shape at a lower concentration. On the other hand, two or more formed dry silk fibroin materials, cannot be bonded to each other or cannot be bonded to other substrate materials after treatment with the above inducers.

### SUMMARY OF THE INVENTION

To solve the above technical problems, an object of the present invention is to provide use of an aromatic alcohol as a structural inducer for silk fibroin and a method for treating silk fibroin materials. The present invention discloses use of an aromatic alcohol as a structural inducer for silk fibroin, which provides a new direction for the development of reagents that induce changes in the secondary structure of silk fibroin.

A first object of the present invention is to provide use of an aromatic alcohol as a structural inducer for silk fibroin.

Preferably, the aromatic alcohol is selected from the group consisting of the compounds having a structure below:

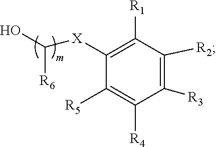

wherein m=0-5; X is methylene or oxygen; and R₁, R₂, R₃, R₄, R₅, and R₆ are each independently selected from hydrogen (—H), phenyl, hydroxyl (—OH), carboxyl (—COOH), aldehyde group (—CHO), keto (—CO—), ester group (—COO—), ether group (—O—), amino (—NH₂), nitro (—NO₂), cyano (—CN), amido (—CO—NH₂), azo (—N═N—), amidino (HN═C(NH₂)—), oximido, a hydrazone group, halo (—X), an acid halide (—CO—X), a sulfonic acid group (—SO₃H), a disulfide bond (—S—S—), sulfydryl (—SH), phosphino (—PH₂), and a phosphate group (—PO₃H₂).

Preferably, the aromatic alcohol has a structure of

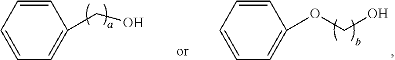

in which a=1-5, and b=1-5.

Preferably, the aromatic alcohol is in a liquid or vapor state.

Preferably, the aromatic alcohol is selected from the group consisting of benzyl alcohol, phenethyl alcohol, phenylpropanol, phenylbutanol, phenylpentanol, 2-phenoxyethanol and any combination thereof.

A second object of the present invention is to provide a treatment method for obtaining a water-insoluble silk fibroin, which comprises the following steps:

bringing silk fibroin into contact with an aromatic alcohol-containing vapor or liquid to induce the structural transformation of silk fibroin, so as to obtain the water-insoluble silk fibroin.

Preferably, the aromatic alcohol is selected from the group consisting of the compounds having a structure below:

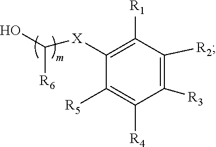

in which m=0-5; X is methylene or oxygen; and R₁, R₂, R₃, R₄, R₅, and R₆ are each independently selected from hydrogen (—H), phenyl, hydroxyl (—OH), carboxyl (—COOH), an aldehyde group (—CHO), keto (—CO—), an ester group (—COO—), an ether group (—O—), amino (—NH₂), nitro (—NO₂), cyano (—CN), amido (—CO—NH₂), azo (—N═N—), amidino (HN═C(NH₂)—), oximido, a hydrazone group, halo (—X), an acid halide (—CO—X), an sulfonic acid group (—SO₃H), a disulfide bond (—S—S—), sulfydryl (—SH), phosphino (—PH₂), and a phosphate group (—PO₃H₂).

Preferably, the aromatic alcohol has a structure of

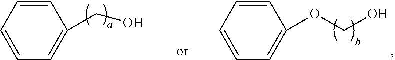

in which a=1-5, and b=1-5.

Preferably, the aromatic alcohol is selected from the group consisting of benzyl alcohol, phenethyl alcohol, phenylpropanol, phenylbutanol, phenylpentanol and 2-phenoxyethanol.

Preferably, the silk fibroin is in a dry state and has a moisture content of not higher than 30%. If the treatment method of the present invention is applied to a formed dry silk fibroin material, the treated silk fibroin has good viscosity, thereby improving its adhesion to other silk fibroin materials or substrates.

Preferably, the silk fibroin is soaked in an aromatic alcohol-containing liquid or the silk fibroin is fumigated with an aromatic alcohol-containing vapor.

Preferably, when an aromatic alcohol-containing liquid is used for treatment, the treatment time is not less than 5 seconds; and the treatment temperature is 20-200° C., and more preferably, 20-120° C.

Preferably, when an aromatic alcohol-containing vapor is used for treatment, the treatment time is not less than 5 min, and the treatment temperature is 25-200° C., and more preferably, 20-120° C.

Preferably, the aromatic alcohol-containing vapor or liquid also comprises water and a conventional alcohol inducer such as methanol and ethanol.

In the present invention, when an aromatic alcohol-containing vapor or liquid is used for inducing the silk fibroin, not only a neat silk fibroin material, but also a composite materials consisting of silk fibroin and an additional material can be obtained. The additional material includes: inorganic metal materials such as ceramics, glass and cement; polymer materials such as rubber, chemical fibers and plastics; and structural and functional composite materials. The resulting products are applicable to the fields of medicine, environment, textile, light industry and medical instruments.

In the present invention, “inducer” means a chemical reagent that can transform a water-soluble silk protein material into a water-insoluble silk protein material. The secondary structure of silk fibroin molecules in the former material is dominated by disordered and random coils. In the latter material, the secondary structure of silk fibroin is dominated by ordered α-helices or β-sheets.

By means of the above technical solutions, the present invention has the following advantages.

The present invention provides new use of an aromatic alcohol as a structural inducer for silk fibroin, which provides a new direction for the development of new structural inducers for silk fibroin.

The present invention also provides a treatment method for obtaining a water-insoluble silk fibroin, wherein an aromatic alcohol-containing vapor or liquid is used for treatment. The method has simple process, the treated silk fibroin is insoluble in water, and has a higher mechanical strength than a product obtained by a traditional treatment method. After treatment with the inducer of the present invention, the mechanical performance of the silk fibroin material and the bonding strength between the silk fibroin material and a substrate material are over 50% higher than those of a silk fibroin material obtained by a traditional treatment method (induction with methanol, ethanol, and steam).

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 shows the chemical formulas, structural formulas, molecular weights and saturated vapor pressures of various reagents.

FIG. 2 shows the weight change of a silk fibroin film after being soaked with different reagents, including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 3 shows the weight change of a silk fibroin film after being soaked or fumigated with different reagents, including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol;

FIG. 4 shows the change in secondary structure after a silk fibroin film is soaked and fumigated with different reagents, including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol;

FIG. 5 shows the breaking strength of a silk fibroin film after being fumigated at 25, 60 and 90° C. with different reagents, including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 6 shows the surface morphology of a silk fibroin film after treatment with different vapors at 25° C., including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 7 shows the surface morphology of a silk fibroin film after treatment with different vapors at 60° C., including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 8 shows the surface morphology of a silk fibroin film after treatment with different vapors at 90° C., including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 9 shows the test results of the bonding strength between silk fibroin films after being soaked or fumigated with different reagents, including (A) Water, (B) Methanol, (C) Benzyl alcohol, (D) Ethanol, (E) Phenethyl alcohol, (F) 2-Phenoxyethanol, (G) 3-Phenyl-1-propanol, (H) 4-Phenylbutanol, (I) 1,3-Butanediol.

FIG. 10 shows the bonding strength of a silk fibroin film after being fumigated with ethanol and phenethyl alcohol at 60° C. for 1, 3, 6 and 12 hrs.

FIG. 11 shows the bonding strength of a silk fibroin film with smooth and ground glass after being fumigated at 60° C. with ethanol and phenethyl alcohol for 3 hrs.

FIG. 12 is a schematic view of a fully enclosed evaporating dish used to suspend chemical fiber fabrics.

FIG. 13 shows photos of surface morphology of chemical fiber fabrics after being soaked and fumigated with a silk fibroin solution for 1, 3 and 5 hrs respectively, in which (a) untreated chemical fiber fabric; (b) chemical fiber fabric soaked with a 5 wt % silk fibroin solution; (c), (f), and (i) chemical fiber fabric soaked with a 5 wt % silk fibroin solution and then fumigated with phenethyl alcohol for 1 hr, 3 hrs and 5 hrs; (d), (g) and (j) chemical fiber fabric soaked with a 1 wt % silk fibroin solution and then fumigated with phenethyl alcohol for 1 hr, 3 hrs and 5 hrs; and (e), (h) and (k) chemical fiber fabric soaked with a 0.1 wt % silk fibroin solution and then fumigated with phenethyl alcohol for 1 hr, 3 hrs and 5 hrs.

FIG. 14 shows the changes in air permeability of chemical fiber fabrics after being soaked and fumigated with a silk fibroin solution for 1, 3 and 5 hrs respectively, in which (a) untreated chemical fiber fabric; (b) chemical fiber fabric soaked with a 5 wt % silk fibroin solution; (c), (f), and (i) chemical fiber fabric soaked with a 5 wt % silk fibroin solution and then fumigated with phenethyl alcohol for 1 hr, 3 hrs and 5 hrs; (d), (g) and (j) chemical fiber fabric soaked with a 1 wt % silk fibroin solution and then fumigated with phenethyl alcohol for 1 hr, 3 hrs and 5 hrs; and (e), (h) and (k) chemical fiber fabric soaked with a 0.1 wt % silk fibroin solution and then fumigated with phenethyl alcohol for 1 hr, 3 hrs and 5 hrs.

FIG. 15 shows the thickness change of chemical fiber fabrics after being soaked and fumigated with a silk fibroin solution for 1, 3 and 5 hrs respectively, in which (a) untreated chemical fiber fabric; (b) chemical fiber fabric soaked with a 5 wt % silk fibroin solution; (c), (f), and (i) chemical fiber fabric soaked with a 5 wt % silk fibroin solution and then fumigated with phenethyl alcohol for 1 hr, 3 hrs and 5 hrs; (d), (g) and (j) chemical fiber fabric soaked with a 1 wt % silk fibroin solution and then fumigated with phenethyl alcohol for 1 hr, 3 hrs and 5 hrs; and (e), (h) and (k) chemical fiber fabric soaked with a 0.1 wt % silk fibroin solution and then fumigated with phenethyl alcohol for 1 hr, 3 hrs and 5 hrs.

FIG. 16 shows the changes in moisture regain of chemical fiber fabrics after being soaked and fumigated with a silk fibroin solution for 1, 3 and 5 hrs respectively, in which (a) untreated chemical fiber fabric; (b) chemical fiber fabric soaked with a 5 wt % silk fibroin solution; (c), (f), and (i) chemical fiber fabric soaked with a 5 wt % silk fibroin solution and then fumigated with phenethyl alcohol for 1 hr, 3 hrs and 5 hrs; (d), (g) and (j) chemical fiber fabric soaked with a 1 wt % silk fibroin solution and then fumigated with phenethyl alcohol for 1 hr, 3 hrs and 5 hrs; and (e), (h) and (k) chemical fiber fabric soaked with a 0.1 wt % silk fibroin solution and then fumigated with phenethyl alcohol for 1 hr, 3 hrs and 5 hrs.

FIG. 17 shows photos of silk fibroin modified fibers and textiles based on silk fibroin modified fibers.

### DETAILED DESCRIPTION OF THE PREFERRED EMBODIMENTS

The specific embodiments of the present invention will be described in further detail with reference to embodiments. The following embodiments are intended to illustrate the present invention, instead of limiting the scope of the present invention.

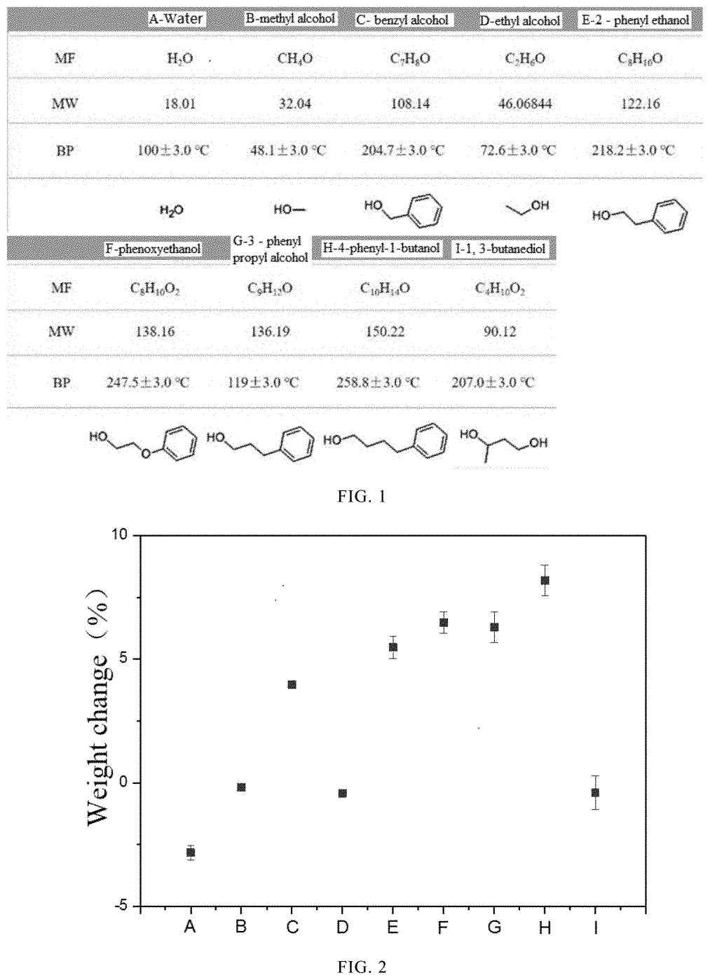

FIG. 1 illustrates the chemical formulas, molecular weights, saturated vapor pressures and structural formulas of various reagents. The saturated vapor pressure in the figure is the vapor pressure under a pressure of 760 mmHg column.

### EXAMPLE 1: PREPARATION OF REGENERATED SILK FIBROIN

(1) 250 g of raw silkworm silk was weighed, and cut into segments having a length of about 15 cm each. 100 liters of pure water was heated by an electric heating tank, and 212 g of anhydrous sodium carbonate was slowly added when the water was nearly boiling, stirred until uniform, and continuously heated to boiling. Then raw silk was added, and timing was started when the raw silk was all submerged in the water. The raw silk was stirred every 7 min, and taken out after 30 min. The degummed silk was fed to a washing machine, and rinsed twice with pure water, following a set rinsing procedure. The dehydrated degummed silk was fed to an air dry oven, and dried for 12 to 18 hrs.

(2) A 9.3 M lithium bromide solution was prepared, and according to a bath ratio of the degummed silk to the lithium bromide solution of 1/4, the degummed silk was added. The degummed silk was dissolved in a dry oven at 60° C. for 4 hrs, taken out, and cooled to room temperature. The dissolved silk fibroin solution was added to a dialysis bag with a molecular weight cut-off of 3500, and dialyzed against pure water for 48 hrs, during which the water was changed 6 times. The dialyzed silk fibroin solution was filtered with 5 layers of gauze to remove impurities, and stored in a refrigerator at 2-8° C. for later use.

### EXAMPLE 2: PREPARATION OF SILK FIBROIN FILM AND TEST OF MOISTURE CONTENT

(1) The silk fibroin solution obtained in Example 1 was diluted to a content of 4% by weight, and 2 mL of the solution was added to a plastic petri dish with a diameter of 3.5 cm. The solution was stood for more than 3 hrs, until the internal air bubbles were completely removed. Then, the solution was placed in a fume hood at room temperature, and air dried overnight to form a film. The moisture content (W %) of the silk fibroin film was determined by weight loss method:

W %=(W₁−W₂)/W₁*100% (1)

where W₁ represents the initial dry weight of the newly prepared silk fibroin film, and W₂ represents the weight of the silk fibroin film after thorough drying (at 120° C. for 12 hrs). The experiment was repeated 5 times to improve the reliability of the experiment.

Experimental results show that the internal moisture content is about 3.64%, after the silk fibroin film is naturally air-dried for 12 hrs at room temperature.

### EXAMPLE 3: TREATMENT OF SILK FIBROIN FILMS WITH LIQUIDS OR VAPORS CONTAINING DIFFERENT REAGENTS AND CHARACTERIZATION OF CHANGES IN THEIR MECHANICAL PERFORMANCE

(1) The silk fibroin films with a moisture content of about 3.64% prepared in Example 2 were respectively soaked in water, methanol, benzyl alcohol, ethanol, phenethyl alcohol, 2-phenoxyethanol, 3-Phenyl-1-propanol, 4-phenylbutanol and 1,3-butanediol, for 5 min at room temperature. Then the silk fibroin film soaked in each reagent was taken out, the remaining reagent on the surface was absorbed with filter paper, and the resulting sample was weighed. The weight change of the sample after treatment is shown in FIG. 2. In FIGS. 2, A, B, C, D, E, F, G, H, and I represent water, methanol, benzyl alcohol, ethanol, phenethyl alcohol, 2-phenoxyethanol, 3-Phenyl-1-propanol, 4-phenylbutanol, and 1,3-butanediol, respectively. Unless otherwise indicated, the letters in the following drawings have the same meaning as defined here. The results show that the silk fibroin film treated with ethanol has almost no change in weight; and the silk fibroin film treated with an aromatic alcohol has a weight increase associated with the length of the carbon chain. This may be due to the fact that the aromatic alcohol has a high viscosity and is adhered to the surface of the silk fibroin film material, which causes weight increase of the material.

(2) Then, the silk fibroin films of the present invention soaked in various alcohols were washed with ethanol to remove the non-specifically bound aromatic alcohol, and then washed with deionized water to remove residual ethanol. The sample was placed in a fume hood and dried overnight at room temperature.

The dried silk fibroin films of the above groups were dried, the dry weight W₃ of each silk fibroin film was determined, and the weight change rate (Wₘ %) of each group of sample was calculated from the weights of the sample before and after treatment.

Wₘ %=(W₁−W₃)/W₁*100% (2)

where W₁ represents the initial dry weight of the silk fibroin film in Example 2. The experiment was repeated 5 times to improve the reliability of the experiment.

(3) The silk fibroin films with a moisture content of about 3.64% prepared in Example 2 were respectively placed on an upper layer of a specialized evaporating dish, and the lower layer was a certain amount of water, methanol, benzyl alcohol, ethanol, phenethyl alcohol, 2-phenoxyethanol, 3-Phenyl-1-propanol, 4-phenylbutanol and 1,3-butanediol respectively. The temperature of each group of experiments was set to 60° C., and the time was 3 hrs. Under the above conditions, the various reagents became a vapor, and were brought into contact with the silk fibroin films in the form of vapors.

(4) The silk fibroin film treated by the above method was taken out, the remaining reagent on the surface was removed by blow drying with an air blower, and the silk fibroin film was weighed immediately. The dry weight W₄ of each silk fibroin film was determined, and The moisture content (W %) of the silk fibroin film was determined by weight loss method:

Wₙ %=(W₁−W₄)/W₁*100% (3)

where W₁ represents the initial dry weight of the silk fibroin film in Example 2. The experiment was repeated 5 times to improve the reliability of the experiment.

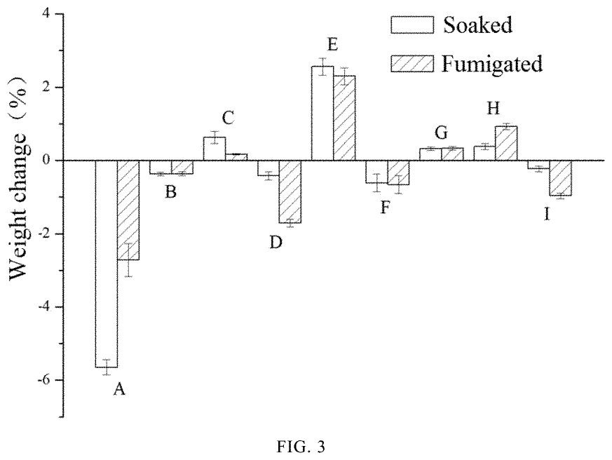

FIG. 3 shows the weight changes of silk fibroin films treated by the above two methods (soaking or fumigation). It can be seen that After being soaked in each reagent, the silk fibroin film treated with an aromatic alcohol has a weight increase, in which the weight increase of the sample treated with phenethyl alcohol reaches a maximum of 2.56%. The weight of the sample treated with ethanol in the control group is reduced by a rate of 0.42%, possibly due to the loss of a small amount of low-molecular-weight silk protein in the film with ethanol soaking and washing. In other control groups including water, methanol, and 1,3-butanediol group, the weight decreases. The weight change of the sample after fumigation with each reagent is slightly different from that after the soaking treatment, but the overall trend is consistent with the soaking treatment.

Further, the performance of the silk fibroin film after the above treatment was characterized as follows.

A. According to the test standard, the silk fibroin film treated with a liquid or vapor of different reagents in Step (1) and Step (3) was cut into a silk fibroin film of a certain area, and scanned for the wave number by an infrared spectrometer in a range of 400-4000 cm⁻¹.

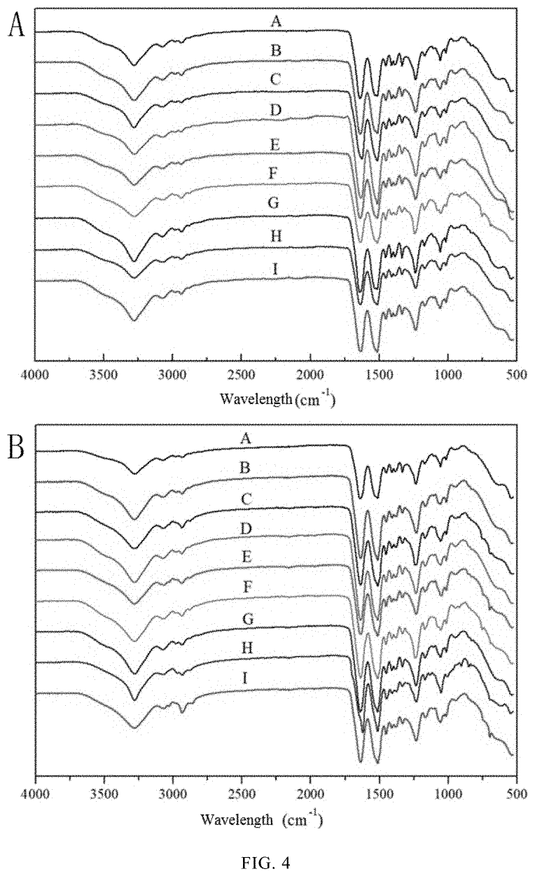

FIGS. 4a and b shows the test results by infrared spectrometry of silk fibroin films treated by soaking and fumigating with different reagents. It can be seen that after being soaked in each reagent, the secondary structure of the silk fibroin film treated with an aromatic alcohol undergoes a certain transformation, in which the characteristic absorption peaks of the silk fibroin film treated with phenethyl alcohol was observed to shift to 1615-1635 cm⁻¹, and 1525-1541cm⁻¹. The silk fibroin film treated with other aromatic alcohols, such as 2-phenoxyethanol, 3-Phenyl-1-propanol and 4-phenylbutanol, has consistent characteristic absorption peaks with those of the silk fibroin film treated with phenethyl alcohol, indicating that the aromatic alcohols have the same mechanism of action on silk fibroin film. Although traditional inducers such as water, ethanol and methanol can also induce the characteristic absorption peak of silk fibroin film to shift, the sharpness of the absorption peak is weaker than that of the silk fibroin film treated with an aromatic alcohol. This shows that the degree of secondary structure (β-sheet) transformation is lower than that of the silk fibroin film treated with an aromatic alcohol. It is generally believed that as the β-sheet content in a silk fibroin material increases, the mechanical performance of the material increases. The above results indicate that aromatic alcohols have obvious advantages in the induction of silk fibroin films.

B. Considering the different boiling points and saturated vapor pressures of different reagents, the influence of fumigation temperature on the induction of silk fibroin films was further investigated. In the present invention, the silk fibroin films prepared in Example 2 were respectively fumigated with water, methanol, benzyl alcohol, ethanol, phenethyl alcohol, 2-phenoxyethanol, 3-Phenyl-1-propanol, 4-phenylbutanol, and 1,3-butanediol and other reagents. The fumigation temperature was 25° C., 60° C. and 90° C., and the fumigation time was 2 hrs.

According to the test standard, the silk fibroin film treated following the above method was prepared into samples, and the breaking strength of the silk fibroin film was tested by Instron-3365 testing machine.

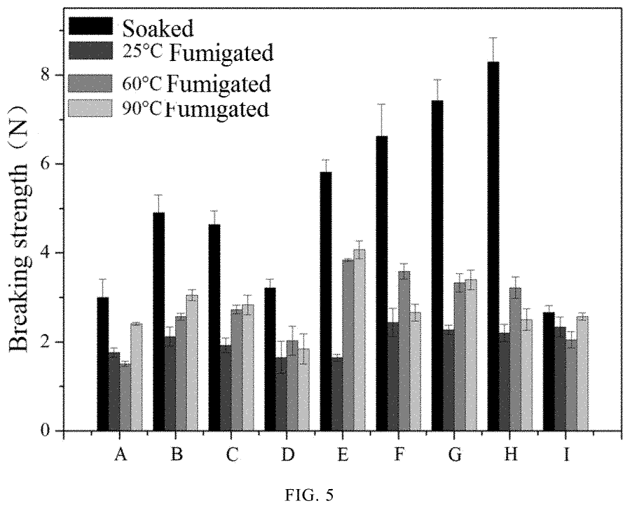

FIG. 5 shows the breaking strength of the silk fibroin film treated following the above method by fumigating with a vapor of water, methanol, benzyl alcohol, ethanol, phenethyl alcohol, 2-phenoxyethanol, 3-Phenyl-1-propanol, 4-phenylbutanol and 1,3-butanediol at 25° C., 60° C., and 90° C. In the control group, the silk fibroin film was soaked with a reagent such as water, methanol, ethanol, phenethyl alcohol, 2-phenoxyethanol, 3-Phenyl-1-propanol, 4-phenylbutanol and 1,3-butanediol. It can be seen from FIG. 5 that the breaking strength of the silk fibroin film treated with an aromatic alcohol increases with the increase of the length of carbon-carbon branch chain. Except that the breaking strength of the silk fibroin film treated with benzyl alcohol is slightly lower than the breaking strength of the silk fibroin film treated with methanol, the breaking strength of the silk fibroin films treated with other aromatic alcohols is higher than that after treatment with non-aromatic alcohol reagents. Further, for silk fibroin films obtained by soaking and fumigating at different temperatures with the same reagent, it is found that the breaking strength after the former treatment is higher than the latter three cases, indicating that soaking is conducive to a sufficient chemical reaction of the silk fibroin film. As the temperature rises, the strength of the silk protein film after fumigation is obviously enhanced. The breaking strengths of silk fibroin films treated at the fumigation temperatures of 60° C. and 90° C. are close, suggesting that if the silk fibroin film is treated by fumigation, it has little effect on the change of mechanical performance after the temperature exceeds 60° C.

C. The silk fibroin films treated with vapors of different reagents at 25° C., 60° C. and 90° C. were respectively adhered to a round platform of an electron microscope, and coated with a metal (10 mA, 90 s). At a voltage of 3 kV and a current of 10 μA, the surface topography was observed by cold-field-emission scanning electron microscopy, to determine the influence of different reagents on the surface morphology of silk fibroin.

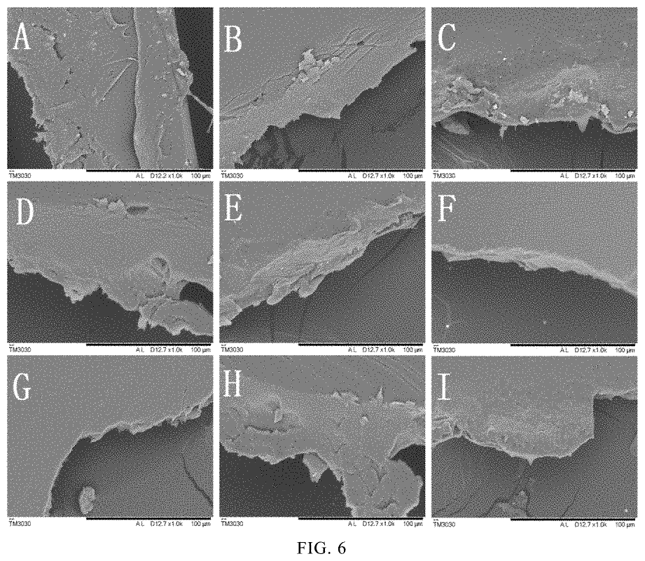

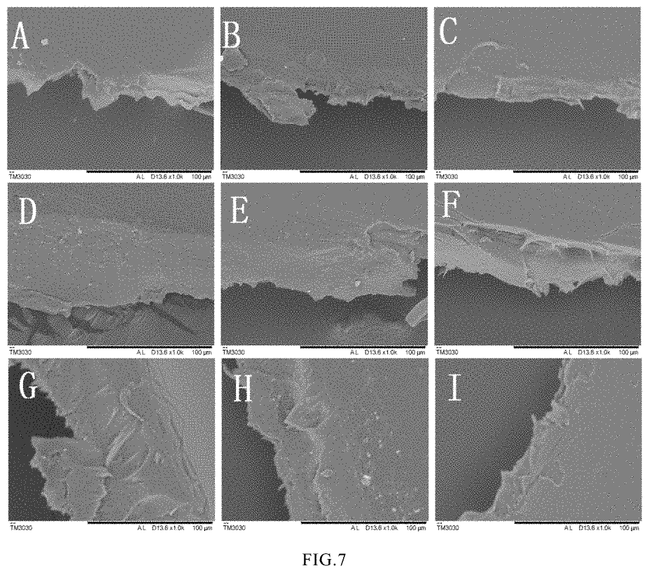

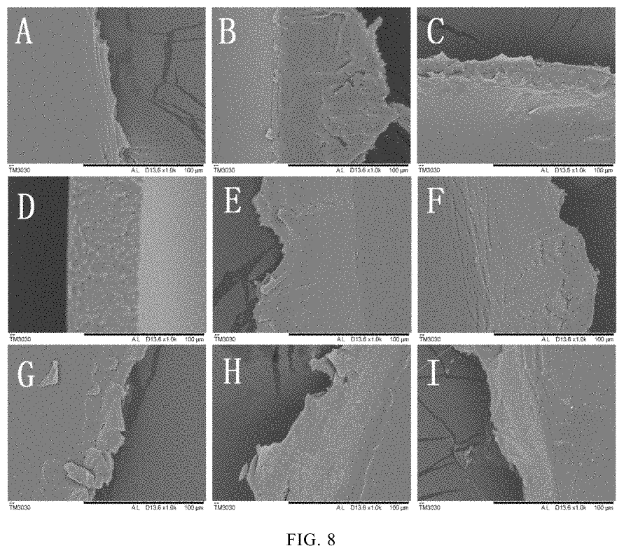

FIGS. 6 to 8 respectively show the surface morphology of silk fibroin films after treatment with different vapors at 25° C., 60° C., and 90° C. The letter numbers in the figures have the same meaning as those in FIG. 2. It can be seen from FIGS. 6 to 8 that the treatment with different reagents does not have influence on the surface morphology of the silk fibroin film, the surface of the silk fibroin film is flat, and no voids, holes and particles are observed.

### EXAMPLE 4: CHANGES IN BONDING FORCE BETWEEN DRY SILK FIBROIN FILMS AFTER TREATMENT WITH VAPORS OF DIFFERENT REAGENTS

(1) 2 silk fibroin films with a moisture content of about 3.64% prepared in Example 2 were laminated, placed horizontally, and pressed by a counterweight of 500 g, to laminate them together. Multiple groups of parallel experiments were performed, each group having at least two silk fibroin films. The silk fibroin film in each group was respectively soaked in water, methanol, benzyl alcohol, ethanol, phenethyl alcohol, 2-phenoxyethanol, 3-Phenyl-1-propanol, 4-phenylbutanol and 1,3-butanediol for 3 hrs, at room temperature.

(2) 2 silk fibroin films with a moisture content of about 3.64% prepared in Example 2 were laminated, placed horizontally, and pressed by a counterweight of 500 g, to laminate them together. Multiple groups of parallel experiments were performed, each group having at least two silk fibroin films. The silk fibroin film in each group was respectively fumigated with water, methanol, benzyl alcohol, ethanol, phenethyl alcohol, 2-phenoxyethanol, 3-Phenyl-1-propanol, 4-phenylbutanol and 1,3-butanediol at 60° C. for 3 hrs.

(3) The same group of silk fibroin films treated in the Steps (1) and (2) were laminated to each other, and a sample was prepared according to the test standard. The maximum bonding strength upon separation from each other was tested by the Instron-3365 testing machine to determine the specificity of different reagents in the treatment of silk fibroin, and distinguish the effect of reagents with or without a phenyl group on the bonding strength of silk fibroin.

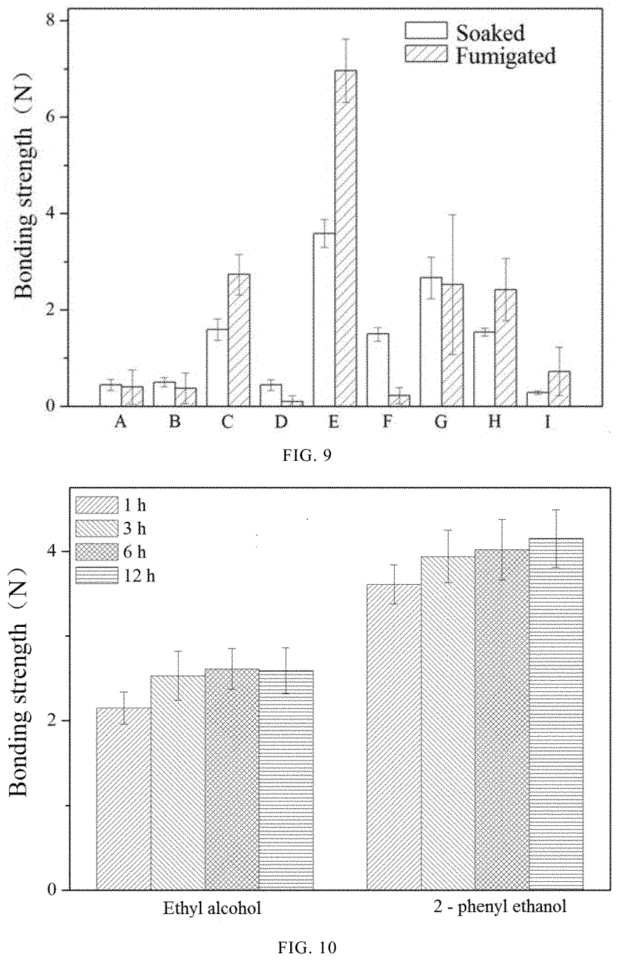

FIG. 9 shows the test result of bonding strength after the silk fibroin film is soaked or fumigated with water, methanol, benzyl alcohol, ethanol, phenethyl alcohol, 2-phenoxyethanol, 3-Phenyl-1-propanol, 4-phenylbutanol and 1,3-butanediol. It can be seen from FIG. 9 that the bonding force between the silk fibroin films after treatment with an aromatic alcohol is higher than the bonding force between the silk fibroin films after treatment with a reagent without a phenyl group. The bonding force is the largest in the treatment group with phenethyl alcohol, and much higher than groups with other alcohols. This is because obvious interaction exists between the aromatic alcohol and the silk fibroin material, and phenethyl alcohol can be bound to silk fibroin materials with high strength due to the specific conformational relationship between the phenyl and the hydroxyl to form tight bridging sites between silk fibroin molecules thereby achieving a strong bonding effect. Further, due to the high permeability and high infiltrability of gas molecules, there are more binding sites between alcohol and silk protein in the vapor-treated film than in the film soaked with a liquid, and the sites are more evenly distributed, leading to a higher bonding force.

### EXAMPLE 5: INFLUENCE OF FUMIGATION TIME ON BONDING FORCE BETWEEN DRY SILK FIBROIN FILMS

(1) The silk fibroin films with a moisture content of about 3.64% prepared in Example 2 were mechanically clamped, and forced to be laminated together by an external force. The silk fibroin films were fumigated with ethanol (control) or phenethyl alcohol at 60° C. for 1, 3, 6 and 12 hrs. Each group had at least two silk fibroin films.

(2) The same group of silk fibroin films treated in the above step were laminated to each other, and a sample was prepared according to the test standard. The maximum bonding strength upon separation from each other was tested by the Instron-3365 testing machine to determine the specificity of different alcohol reagents in the treatment of silk fibroin, and distinguish the effect of reagents with or without a phenyl group on the bonding strength of silk fibroin.

FIG. 10 shows the bonding strength of a silk fibroin film after fumigating with ethanol and phenethyl alcohol at 60° C. for 1, 3, 6 and 12 hrs. The bonding strength between the silk fibroin films after fumigation with phenethyl alcohol is obviously higher than that after fumigation with ethanol, indicating that the former has a potent effect on silk fibroin materials. Further, if the fumigation time exceeds 3 hrs, the bonding strength does not increase any longer, showing that a saturated fumigation effect can be achieved in 3h.

### EXAMPLE 6: TREATMENT WITH VAPORS OF AROMATIC ALCOHOLS PROMOTES THE ADHESION BETWEEN SILK FIBROIN AND DIFFERENT MATERIALS

(1) The silk fibroin solution obtained in Example 1 was added with deionized water stepwise according to the calculated mass fraction to dilute the mass fraction of the solution to 4%.

(2) 2 mL of the solution was added to a plastic petri dish with a diameter of 3.5 cm. The solution was stood for more than 3 hrs, until the internal air bubbles were completely removed. Then, the solution was placed in a fume hood at room temperature, and air dried overnight to form a film.

(3) The silk fibroin film prepared above was respectively clamped with smooth, and frosted glass, and forced to be laminated together by an external force. The samples were fumigated with ethanol (control) or phenethyl alcohol at 60° C. for 3 hrs.

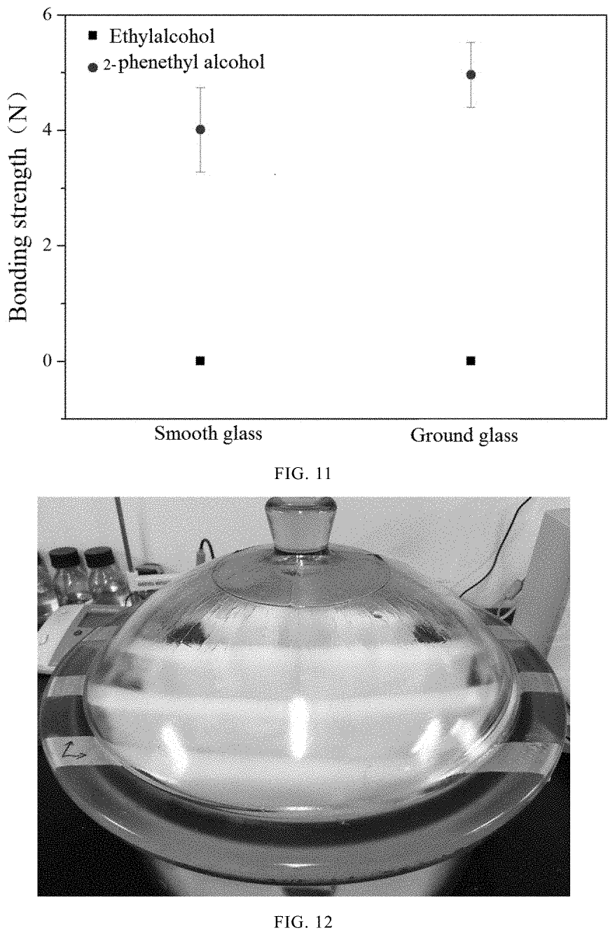

(4) FIG. 11 shows the test results of bonding strength between the silk fibroin film and smooth or frosted glass after being fumigated at 60° C. with ethanol and phenethyl alcohol for 3 hrs following the above method. It can be seen from FIG. 11 that the bonding strength between the silk fibroin film and smooth or frosted glass after treatment with phenethyl alcohol vapor is 4N and 5.1N, respectively. The bonding strength between the silk fibroin film and smooth or frosted glass after treatment with ethanol vapor is 0.1N. Therefore, the bonding strength after treatment with phenethyl alcohol vapor is 40-50 times the adhesion strength after treatment with ethanol vapor.

### EXAMPLE 7: MODIFICATION OF FIBERS WITH SILK FIBROIN AND CHARACTERIZATION OF TEXTILE PROPERTIES (I)

(1) The silk fibroin solution obtained in Example 1 was added with deionized water stepwise according to the calculated mass fraction to dilute the mass fraction of the solution to 0.1-5%.

(2) Polyester, polyamide, polyurethane and other common chemical fiber fabrics that are 10 cm in length and width were soaked in the silk fibroin solutions of different mass fractions above, for 5-10 min, and taken out after being completely infiltrated.

(3) The infiltrated chemical fiber fabrics were treated by a double-roll padder, and the pressure of padding was set by a rotary spring to remove excess silk fibroin solution. The fabrics were removed and air dried.

(4) Method 1: A self-made fully enclosed evaporating dish was used, and a phenethyl alcohol solution was held at the bottom. The evaporating dish was placed in an oven, with a temperature set to 45-120° C. After the phenethyl alcohol solution at the bottom was in a vapor state, the treated chemical fiber fabrics were suspended on the opening of the evaporating dish in the horizontal direction, as shown in FIG. 12. The fumigation time was set to 0.5-6 hrs. Distilled water and ethanol were used as a control group.

### EXAMPLE 8: MODIFICATION OF FIBERS WITH SILK FIBROIN AND CHARACTERIZATION OF TEXTILE PROPERTIES (II)

(1) The silk fibroin solution obtained in Example 1 was added with deionized water stepwise according to the calculated mass fraction to dilute the mass fraction of the solution to 0.1-5%.

(2) Polyester, polyamide, polyurethane and other common chemical fiber fabrics that are 10 cm in length and width were soaked in the silk fibroin solutions of different mass fractions above, for 5-10 min, and taken out after being completely infiltrated.

(3) The infiltrated chemical fiber fabrics were treated by a double-roll padder, and the pressure of padding was set by a rotary spring to remove excess silk fibroin solution. The fabrics were removed and air dried.

(4) Method 2: A phenethyl alcohol solution was held inside the iron. Distilled water and ethanol were used as a control group. The chemical fiber fabrics treated in Step (3) was air dried at room temperature, and ironed.

### EXAMPLE 9: MODIFICATION OF FIBERS WITH SILK FIBROIN AND CHARACTERIZATION OF TEXTILE PROPERTIES (III)

(1) The silk fibroin solution obtained in Example 1 was added with deionized water stepwise according to the calculated mass fraction to dilute the mass fraction of the solution to 0.1-5%.

(2) Method 3: The silk fibroin solutions of different mass fractions obtained in Step (1) were mixed with a phenethyl alcohol solution at a volume ratio of 50:1 to 1:1, and the chemical fiber fabrics were soaked in the above mixed solutions for 5-10 min, and taken out after being completely infiltrated.

(3) The infiltrated chemical fiber fabrics were treated by a double-roll padder, and the pressure of padding was set by a rotary spring to remove excess silk fibroin solution. The fabrics were removed and air dried. The treated chemical fiber fabrics was directly ironed with an iron (containing water therein).

The chemical fiber fabrics prepared by the above three methods were tested for surface morphology, air permeability, fabric thickness and moisture regain. 1) Surface morphology test: The chemical fiber fabrics prepared above were adhered onto a round platform of an electron microscope, and coated with a metal (10 mA, 90 s). At a voltage of 3 kV and a current of 10 μA, the surface topography was observed by cold-field-emission scanning electron microscopy. 2) Air permeability test: The chemical fiber fabrics prepared above were cut into a round shape of 20 cm², and tested by YG461E-III automatic air permeability tester, set to have the following parameters: equilibration time 24 hrs, pressure 100 Pa, temperature 20° C., humidity 65% unit: mm/s. 3) Fabric thickness test: The chemical fiber fabrics prepared above were cut into a round shape of 20 cm², and tested by YG141D-II fabric thickness meter, set to have the following parameters: equilibration time 24 hrs, test standard GB/T 3820-1997, test time 10 s, single cycle, temperature 20° C., humidity 65% unit: mm; 4) Moisture regain test: The chemical fiber fabrics prepared above were cut into a round shape of 20 cm², and tested by HF-MS moisture regain tester, set to have the following parameters: equilibration time 24 hrs, temperature 104° C., measurements 10, test standard GB/T 9995-1997, test time 10 s, single cycle, temperature 20° C., humidity 65% unit %.

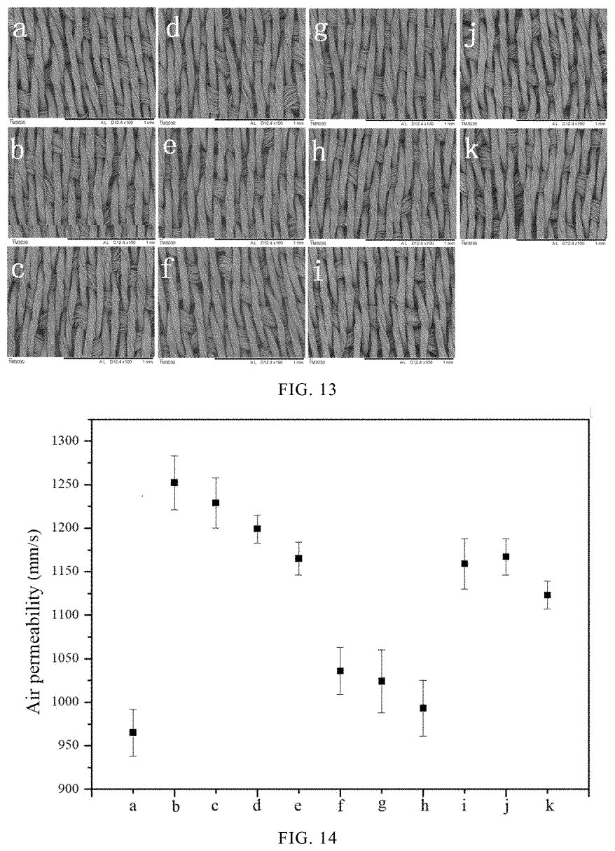

FIG. 13 shows the test results of surface morphology of chemical fiber fabrics after being treated following the method in Example 7. FIG. 13(a) untreated chemical fiber fabric; (b) chemical fiber fabric soaked with a 5 wt % silk fibroin solution for 1 min; (c), (f), and (i) chemical fiber fabric soaked with a 5 wt % silk fibroin solution for 1 min and then fumigated with phenethyl alcohol for 1, 3 and 5 hrs; (d), (g) and (j) chemical fiber fabric soaked with a 1 wt % silk fibroin solution for 1 min and then fumigated with phenethyl alcohol for 1, 3 and 5 hrs; and (e), (h) and (k) chemical fiber fabric soaked with a 0.1 wt % silk fibroin solution for 1 min and then fumigated with phenethyl alcohol for 1, 3 and 5 hrs. Unless otherwise indicated, the letter numbers in FIGS. 14 to 16 have the same meaning as defined here. The results show that the morphology of fabrics treated under different conditions does not undergo obvious changes, the surface is smooth, and has no particles and lumps. This is consistent with the microscopic morphology of natural silk. The surface morphology of fabrics after being treated following the method in Examples 8 and 9 is the same as described above.

FIG. 14 shows the test results of air permeability of the fabrics treated as described in Example 7. The results show that compared with untreated fabrics, the air permeability of fabrics treated by various methods is improved. The air permeability of the treated chemical fiber fabrics can reach 950-993 mm/s, which is close to the air permeability of natural silk fabrics of 956 mm/s. The air permeability of fabrics after being treated following the method in Examples 8 and 9 is the same as described above.

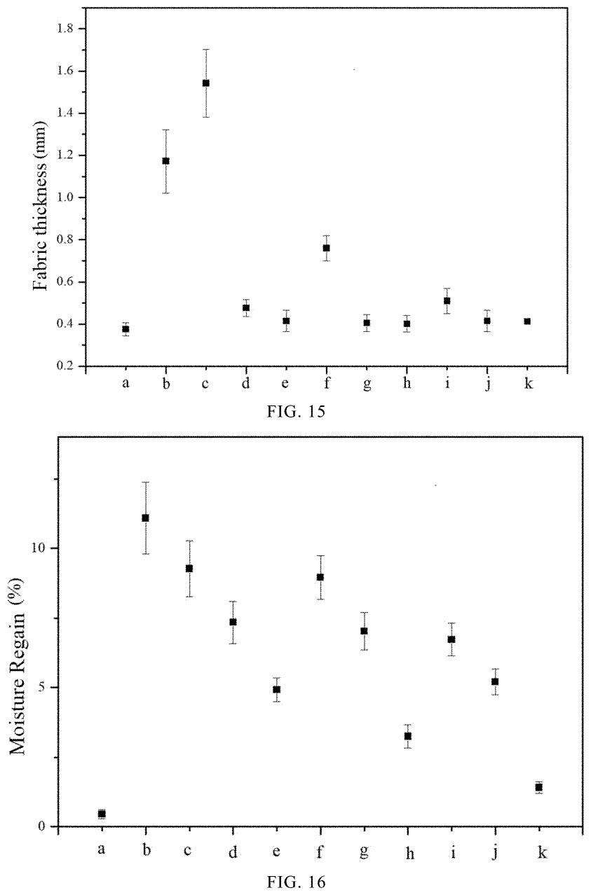

FIGS. 15 and 16 show the test results of fabric thickness and moisture regain of fabrics treated as described in Example 7. The fabric thickness of the treated chemical fiber fabrics can reach 0.40-0.47 mm, which is close to the fabric thickness of the natural silk fabrics of 0.37 mm. The standard moisture regain of the treated chemical fiber fabrics can reach 9.27-11.03%, which is close to the standard moisture regain of natural silk fabrics of 11.9%. The fabric thickness and moisture regain of fabrics after being treated following the method in Examples 8 and 9 are the same as described above.

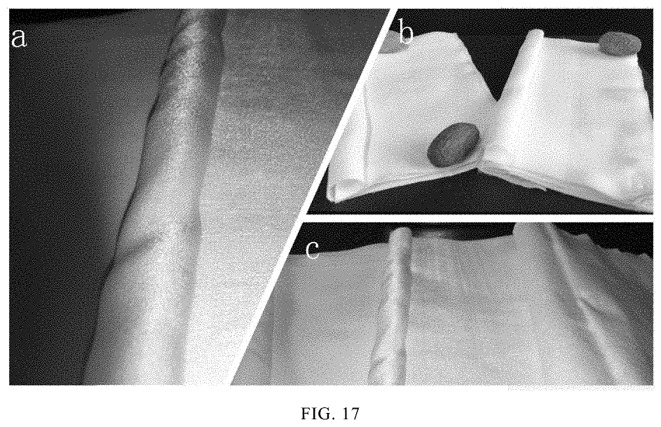

FIG. 17 shows photos of silk fibroin modified fibers and textiles based on silk fibroin modified fibers. The results show that the treated silk fibroin has good glossiness.

The above results show that the chemical fiber fabrics treated by the above three methods have a silk imitation rate of up to 95-99%.

While preferred embodiments of the present invention have been described above, the present invention is not limited thereto. It should be appreciated that some improvements and variations can be made by those skilled in the art without departing from the technical principles of the present invention which are also contemplated to be within the scope of the present invention.

1. Use of an aromatic alcohol as a structural inducer for silk fibroin.

2. The use according to claim 1, wherein the aromatic alcohol is selected from the group consisting of the compounds having a structure below: 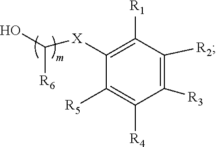
wherein m=0-5;
X is methylene or oxygen; and
R₁, R₂, R₃, R₄, R₅, and R₆ are each independently selected from hydrogen, phenyl, hydroxyl, carboxyl, an aldehyde group, keto, an ester group, an ether group, amino, nitro, cyano, amido, azo, amidino, oximido, a hydrazone group, halo, an acid halide, a sulfonic acid group, a disulfide bond, sulfydryl, phosphino, or a phosphate group.

3. The use according to claim 1, the aromatic alcohol is in a liquid or vapor state.

4. A treatment method for obtaining a water-insoluble silk fibroin material, comprising steps of:
bringing silk fibroin into contact with an aromatic alcohol-containing vapor or liquid to induce the structural transformation of silk fibroin, to obtain the water-insoluble silk fibroin material.

5. The treatment method according to claim 4, wherein the aromatic alcohol is selected from the group consisting of the compounds having a structure below: 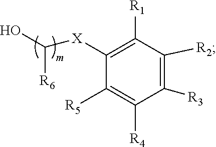
wherein m=0-5;
X is methylene or oxygen; and
R₁, R₂, R₃, R₄, R₅, and R₆ are each independently selected from hydrogen, phenyl, hydroxyl, carboxyl, an aldehyde group, keto, an ester group, an ether group, amino, nitro, cyano, amido, azo, amidino, oximido, a hydrazone group, halo, an acid halide, a sulfonic acid group, a disulfide bond, sulfydryl, phosphino, or a phosphate group.

6. The treatment method according to claim 4, wherein the silk fibroin material is in a dry state and has a moisture content of not higher than 30%.

7. The treatment method according to claim 4, wherein the silk fibroin material is soaked in an aromatic alcohol-containing liquid or the silk fibroin material is fumigated with an aromatic alcohol-containing vapor.

8. The treatment method according to claim 4, wherein when an aromatic alcohol-containing liquid is used for treatment, the treatment time is not less than 5 seconds; and the treatment temperature is 20-200° C.

9. The treatment method according to claim 4, wherein when an aromatic alcohol-containing vapor is used for treatment, the treatment time is not less than 5 min, and the treatment temperature is 25-200° C.

10. The treatment method according to claim 4, wherein the aromatic alcohol-containing vapor or liquid also comprises water and/or an alcohol inducer.
